(19) United States
(12) Patent Application Publication
(10) Pub. No.: US 2007/0149987 A1
(43) Pub. Date: Jun. 28, 2007

(54) Vessel wound closure device

(75) Inventors: Parris Wellman, Reading, MA (US); Bryan Ogle, Louisville, KY (US); Paul Johnson, Cambridge (GB); David Szabo, Doylestown, PA (US); Benjamin Wicks, Huntingdon (GB); Trevor Beckett, Cambridge (GB)

Correspondence Address: SCULLY SCOTT MURPHY & PRESSER, PC, 400 GARDEN CITY PLAZA, SUITE 300, GARDEN CITY, NY, 11530, US

(73) Assignee: Ethicon, Inc., Somerville, NJ (US)

(21) Appl. No.: 11/317,039

(22) Filed: Dec. 22, 2005

(51) Int. Cl. A61B 17/04

(52) U.S. Cl. 606/148

(57) ABSTRACT

A vessel wound closure device that separately deploys a suture and a snare within a vessel to produce a stitch across a puncture site of a vessel. Once deployed within the vessel, the suture is captured within the snare, withdrawn, and then secured to provide a stitch across the puncture site of the vessel. The vessel wound closure device minimizes the amount of material introduced or left within the vessel or between edges of the puncture site, maximizes healing of the puncture site, and renders the vessel amenable to subsequent interventional or therapeutic procedures at or near the same site.

### BACKGROUND OF THE MENTION

1. Field of the Invention

The invention generally relates to a vessel wound closure device. More particularly, the invention relates to a vessel wound closure device for sealing puncture wounds in a blood vessel such as those that result from certain interventional procedures.

2. Related Art

A large number of therapeutic and diagnostic procedures involve the percutaneous introduction of instrumentation into a blood vessel, for example, percutaneous transluminal coronary angioplasty (PTCA). Such procedures most often involve accessing an intended site through the femoral artery. Ideally, closing and healing of the resultant vascular puncture wound successfully completes the procedure.

Traditionally, the application of external pressure to the skin at the entry site of the instrumentation into the patient has been employed to stem bleeding from the wound. A nurse or physician, for example, applies pressure to the wound site until clotting and tissue rebuilding has occurred sufficiently to seal the perforation. In some situations, the external pressure is maintained for an hour or more, during which time the patient is uncomfortably immobilized. Thus patient comfort and physician efficiency are impaired where such external pressure techniques are employed. Moreover, the patient may require immobilization even after the wound is sealed to minimize the risk of wound re-opening.

Additionally, the risk of hematoma exists while bleeding from the vessel occurs. Such hematoma risk continues until sufficient clotting of the wound site occurs. Moreover, external pressure devices, such as femoral compression systems, are often unsuitable for some patients. For example, patients with substantial amounts of subcutaneous adipose tissue, as the skin surface in such patients may be a considerable distance away from the vasculature puncture site. Inaccurate skin compression, and thus less effective wound healing, tends to occur as a result.

U.S. Pat. No. 5,383,896 to Gershony, et al. discloses a device that applies pressure to a puncture site internally for a limited period of time, after which the device is removed. The device in Gershony includes a shaft with an expandable balloon and a guidewire tip at its distal end. The distal end of the device is introduced into a blood vessel through an introducer sheath that is typically used in percutaneous interventional procedures. The balloon is then inflated and withdrawn until the balloon hemostatically engages the inner surface of the blood vessel, after which the introducer sheath is removed. A fixation collar on the shaft applies tension to the balloon for a medically sufficient time and thereafter the balloon is deflated and the entire device is removed from the body.

U.S. Pat. No. 5,645,566 to Brenneman, et al. discloses a device that applies pressure to the outside wall of a punctured blood vessel from a distance using a balloon, a sheet and a foam pad. The pressure applying device is located using a balloon in the vessel (similar to that of Gershony) and a radiopaque marker.

PCT Application WO 98/11830, published Mar. 26, 1998, S. Barak, Inventor, discloses various embodiments of an apparatus for hemostasis. Among them is a device that positions an “anchor” against an inner surface of an artery wall and a balloon outside the wall. The balloon is inflated to pinch the artery wall, after which the anchor is withdrawn. The balloon is maintained against the puncture until hemostasis is achieved.

Other arterial closure devices include bioabsorbable materials intended to remain in the body until they are absorbed as in U.S. Pat. Nos. 5,282, 827 and 5,662,681.

U.S. Pat. No. 5,391,183 to Janzen, et al. describes a device that inserts hemostatic material through a tissue channel and against the outside wall of the vessel around the puncture site.

U.S. Pat. No. 5,690,674 to Diaz discloses a biodegradable plug that has two substantially parallel disks joined at their centers by a waist. The plug is positioned so that the distal disk is on the interior wall of the blood vessel, the proximal disk is on the exterior wall, and the waist is in the wound of the vessel wall.

Another known closure devices include U.S. Pat. No. 5,741,223 to Janzen, et al. This '223 patent discloses the placement of a plug to seal a puncture site.

U.S. Pat. No. 5,354,271 to Voda which discloses suture threads with barbed ends, wherein the suture threads are deployed into a vessel and then the barbed ends penetrate through the vessel wall and expand to prevent retraction thereof back into the vessel. The suture threads are then tied or otherwise secured across the puncture site.

U.S. Pat. No. 5,324,306 discloses a mass of hemostatic material pushed against the outside wall of a vessel at a puncture site. Manual pressure is applied to ensure blood flow has stopped.

U.S. Pat. No. 5,868,778 discloses a balloon used in combination with a procoagulant injected at the puncture site in order to seal a puncture site of a vessel.

U.S. Pat. No. 5,792,152 discloses a flexible needle with suture attached thereto that is deployed across a puncture site of a vessel. The flexible needle and suture are introduced into the vessel via an entry lumen, proceed through a U-shaped return lumen, and exit the vessel through an exit lumen. Thereafter the suture is drawn further outward from the vessel and tied or otherwise secured across the puncture site.

U.S. Pat. No. 5,441,517 discloses an anchor inserted into a vessel and urged against an inner wall of the vessel as a collagen plug is deployed externally of the puncture site to expand and fill the tissue tract leading to the puncture site. A filament attaches the plug to the anchor. After emplacement, a tamping member may be used to urge the plug against the external puncture site to help seal the same.

U.S. Patent Publication No. 2004/0006352 discloses an arterial closure device comprising an assembly in which clasp arms, to which a suture is initially secured, are deployed within a vessel. Penetrating members including suture catches are then separately deployed to snag or capture the sutures associated with a respective clasp arm. The sutures are then pulled taught by pulling the penetrating member with suture catches out from the vessel, and then tied or otherwise secured to close the puncture site. Thereafter the assembly is withdrawn from the body.

None of the known art disclose or suggest a vessel wound closure device that separately deploys a suture and snare within a vessel, whereby the suture is captured within the snare, withdrawn, and then tied to produce a suture stitch across the puncture site of the vessel, thereby minimizing the amount of material introduced or left within the lumen of the vessel, maximizing wound healing, and rendering the vessel more amenable to subsequent interventional or therapeutic procedures at or near the same site.

### SUMMARY OF THE INVENTION

Various embodiments of the invention described herein comprise a vessel wound closure device that separately deploys a suture and a snare within a vessel to produce a suture across a puncture site of a vessel. Once deployed within the vessel, the suture is captured within the snare, withdrawn, and then secured to provide a stitch across the puncture site of the vessel. The various embodiments of the vessel wound closure device described herein thus minimize the amount of material introduced or left within the vessel or between edges of the puncture site, maximize healing of the puncture site, and render the vessel amenable to subsequent interventional or therapeutic procedures at or near the same site.

In some embodiments, the vessel wound closure device comprises a sheath, a pair of movable vessel penetrating members associated with the sheath, whereby the vessel penetrating members are moved to penetrate the vessel wall adjacent a puncture site, each penetrating member creating a small hole in the vessel wall adjacent the puncture site. A suture is deployed into the vessel through one of the penetrating members, and a single filament snare loop is deployed into the vessel through the other of the penetrating members. The single filament snare loop includes a pair of strands that extend beyond a proximal end of the sheath, whereby movement of one or both of the strands determines the size of the snare loop within the vessel. The suture is captured by the snare loop within the vessel, and then withdrawn by retracting the respective penetrating members. Thereafter, the suture is tightened by further withdrawing the suture and snare via withdrawal of the respective penetrating members. Once tightened as desired, the suture is tied, or otherwise secured, externally of the puncture site so as to create a stitch across the puncture site. Excess suture material is then cut and the wound closure device and its components removed from the patient. In some instances, the sheath of the vessel wound closure device is inserted through an introducer already in place from a preceding procedure. In other instances, the sheath of the vessel closure device is inserted over a guidewire remaining from a preceding procedure, the introducer from the preceding procedure having been removed. Where the introducer from the preceding procedure has been removed and the sheath of the vessel wound closure device is inserted over a guidewire in the vessel, then the location of the vessel penetrating members of the vessel wound closure device is determined by blood flashback through one or both of the respective vessel penetrating members of the device.

A collar, knot or other locking means may also be used to help tighten the suture prior to tying or otherwise securing the suture to form the stitch across the puncture wound of the vessel. Collagen or other thrombogenic material can be incorporated into the locking means to aid hemostasis at the puncture site. The collar, knot or other locking means is preferably bioabsorbable to accommodate resorption thereof in vivo. The collagen or other thrombogenic material may include therapeutic agents such as procoagulants, antimicrobial agents, anesthetics, or the like.

In other embodiments, the vessel wound closure device comprises a body, a first movable vessel penetrating member associated with the body and a second vessel penetrating member associated with the body, a suture slidably housed within one of the vessel penetrating members and a snare slidably housed within the other of the vessel penetrating members, whereby the first and second vessel penetrating members are movable relative to one another and the body so as to penetrate the vessel wall adjacent a puncture site, each vessel penetrating member creating a small hole in the vessel wall adjacent the puncture site. The body has a proximal end and a distal end and further comprises a first exit port at a distal end thereof through which a first vessel penetrating member is deployed, a second exit port at the distal end thereof through which a second vessel penetrating member is deployed, a movable stabilizer foot at the distal end thereof which foot is deployable upon entry of the distal end of the body sufficiently into the vessel, and a flashback port through which flashback blood escapes to indicate when the distal end of the body is located sufficiently within the vessel to deploy the stabilizer foot and the first and second vessel penetrating members. Separately designated slides control the movement and deployment of the stabilizer foot, the first and second vessel penetrating members, the suture, and the snare. The suture is deployed into the vessel through one of the vessel penetrating members, and the snare loop is deployed into the vessel through the other of the vessel penetrating members. In practice, the suture is captured by the snare loop within the vessel, and then withdrawn by retracting the respective vessel penetrating members. Thereafter, the suture is tightened by further withdrawing the suture and the snare via withdrawal of the respective vessel penetrating members. Once tightened as desired, the suture is tied, or otherwise secured, externally of the puncture site so as to create a stitch across the puncture site. Excess suture material is then cut and the vessel wound closure device and its various components are removed from the patient.

In some embodiments, the vessel wound closure device with the stablizer foot is inserted through an introducer already in place from a preceding procedure. In other cases, the introducer from the preceding procedure has been removed and the vessel wound closure device with the stabilizer foot is inserted into the vessel over a guidewire already in place.

The above and other features of the invention, including various novel details of construction and combinations of parts, will now be more particularly described with reference to the accompanying drawings and claims. It will be understood that the various exemplary embodiments of the invention described herein are shown by way of illustration only and not as a limitation thereof. The principles and- features of this invention may be employed in various alternative embodiments without departing from the scope of the invention.

### BRIEF DESCRIPTION OF THE DRAWINGS

These and other features, aspects, and advantages of the apparatus and methods of the present invention will become better understood with regard to the following description, appended claims, and accompanying drawings where:

FIG. 1 illustrates a schematic depiction of an embodiment of a vessel wound closure device according to the invention.

FIG. 2 illustrates a partial schematic depiction of the vessel wound closure device of FIG. 1 advanced to a vessel according to the invention.

FIG. 3 illustrates advancement of the vessel penetrating members of FIG. 1 according to the invention.

FIG. 4 illustrates advancement of the snare of FIG. 1 according to the invention.

FIG. 5 illustrates advancement and capture of the suture of FIG. 1 according to the invention.

FIG. 6 illustrates retraction of the retraction of the captured suture within the snare of FIG. 1 according to the invention.

FIG. 7 illustrates securement of the suture stitch of FIG. 1 according to the invention.

FIGS. 7a-7c illustrate various other suture arrangements to create the suture stitch according to the invention.

FIGS. 8a and 8b illustrates various options of deploying the snare relative to the suture according to the embodiment of FIG. 1.

FIG. 9 illustrates a schematic depiction of another embodiment of a vessel wound closure device according to the invention.

FIG. 10 illustrates in greater detail the inset A of FIG. 9.

FIG. 11 illustrates a single filament snare loop having a mesh.

### DETAILED DESCRIPTION OF THE INVENTION

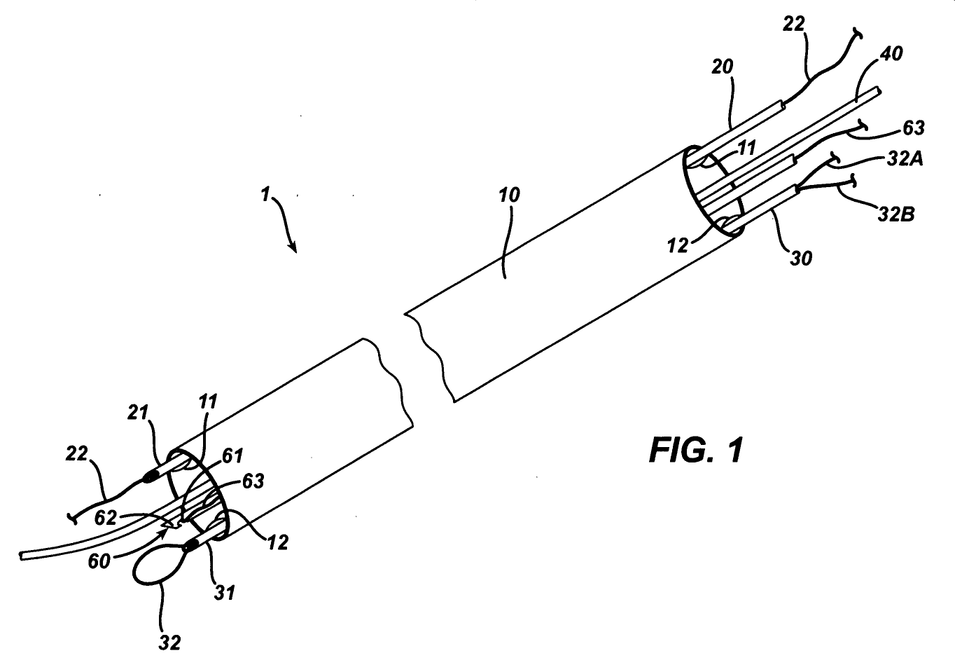

FIG. 1 illustrates one embodiment of a vessel wound closure device 1 according to the invention. The vessel wound closure device 1 comprises a sheath 10 having a distal end and a proximal end, wherein distal is understood as furthest from an operator and proximal is understood as closest to an operator. The vessel wound closure device 1 further comprises a first vessel penetrating member 20 and a second vessel penetrating member 30, each of which is movable relative to the sheath 10. A first guide 11 is preferably provided along an interior surface of the sheath to movably house the first vessel penetrating member 20, and a second guide 12 is preferably provided along an interior surface of the sheath 10 to movably house the second vessel penetrating member 30.

The first guide 11 is preferably opposite the second guide 12, although other arrangements of the first guide 11 relative to the second guide 12 are readily accommodated, in the artisan's discretion. Although FIG. 1 illustrates the first and second vessel penetrating members 20, 30 in guides 11, 12 along an interior surface of the sheath 10, such penetrating members could alternatively be movably housed through guides 11, 12 located on an exterior surface of the sheath 10 as well, in the discretion of the artisan.

The vessel wound closure device 1 of FIG. 1, further comprises a suture 22 extending through the first vessel penetrating member 20, and a single filament snare 32 extending through the second vessel penetrating member 30. As shown in FIG. 1, a distal end of the first vessel penetrating member 20 comprises a pointed needle 21, and a distal end of the second vessel penetrating member 30 comprises a pointed needle 31. The respective needles 21, 31 are movable from a first position to a second position beyond a distal end of the sheath 10 to penetrate through the vessel wall to create holes adjacent a puncture wound when the associated first and second vessel penetrating members 20, 30 are deployed. The suture 22 and the single filament snare 32 are thus positioned in the vessel through the holes created by the respective needles. In practice, the sheath 10 of the vessel wound closure device 1 is placed through an introducer already in place from a preceding procedure, or over a guidewire 40, where the preceding introducer has been removed.

Although the first vessel penetrating member 20 and the second vessel penetrating member 30 are shown as generally circular in cross-section, the artisan should readily appreciate that other cross-sectional configurations are contemplated by the various embodiments described herein. For example, an oval cross-section of either or both of the first and second vessel penetrating members may help assure a more reliable orientation of the snare 32 or suture 22 relative to one another when deployed within the vessel.

Further, either or both of the suture 22 and snare 32 could be pre-bent to help orient the suture and snare relative to one another when deployed within the vessel. The materials comprising the suture and snare may be, for example, a polypropylene, a shape memory alloy, or a stainless/coated steel, in the various embodiments described herein. Ideally, where a shape memory alloy is used to comprise a pre-bent suture or snare. The suture 22 may further comprise knots 22A, or other projections, to assist the capturing of the suture 22 by the single filament snare 32. Moreover, the size of the loop of the single filament snare 32 within the vessel is readily increased or decreased by the medical practitioner by movement of a snare strand 32A extending beyond a proximal end of the wound closure device 1 while maintaining another snare strand 32B in place, the other snare strand 32B also extending beyond the proximal end of the wound closure device.

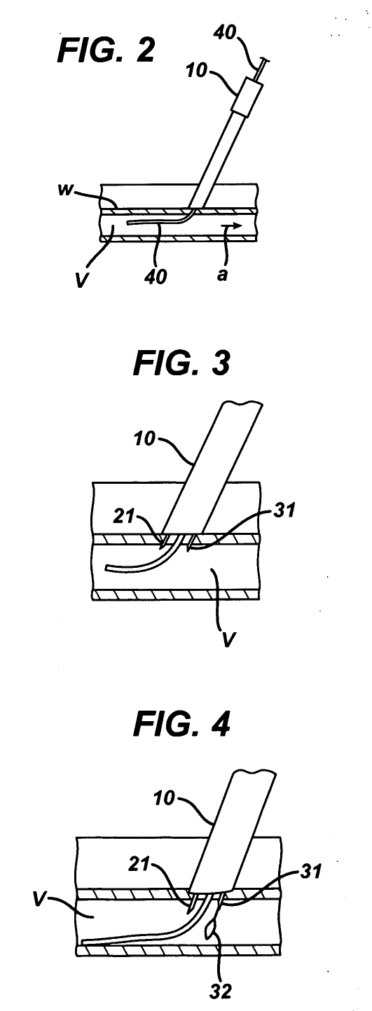

FIGS. 2-7 illustrate various stages of deployment of the suture 22 and the single filament snare 32 and the creation of a stitch across the puncture wound site of a vessel using the embodiment of the vessel wound closure device 1 of FIG. 1, wherein emplacement and use of the vessel wound closure device 1 is generally the same whether it is through an introducer from a preceding procedure, or over a guidewire where the preceding introducer has been removed, unless otherwise specified herein.

In particular, FIG. 2 illustrates a partial schematic view of a distal portion of the vessel wound closure device 1 of FIG. 1 emplaced and used in a vessel V having blood flow proceeding in the direction of arrow a. The blood flow direction of that shown in FIGS. 2-7 is understood to be in the direction of arrow a of FIG. 2, even where arrow a is not otherwise explicitly shown. As shown in FIG. 2, the sheath 10 is inserted through the incision tract over the guidewire 40, for example, and to the vessel wall w. The sheath 10 is preferably oriented such that the second vessel penetrating member is located in the downstream direction of the blood flow so that, when deployed, the single filament snare 32 is downstream of the suture 22, thus rendering capture of the suture 22 by the snare 32 more reliable.

In FIG. 3, the needles 21, 31 of the respective first vessel penetrating member 20 and the second vessel penetrating member 30 are deployed through the vessel wall w, so as to create holes adjacent the puncture wound site of the vessel V. Thereafter, in FIG. 4, the single filament snare 32 is advanced beyond the needle 31 of the second vessel penetrating member 30 and into the vessel V.- As mentioned above, the snare 32 is deployed downstream of the first vessel penetrating member 20 and the suture 22 to be deployed therefrom. The size of the loop of the snare 32 within the vessel V may be increased or decreased by movement of one or both of the strands 32A, 32B extending beyond the proximal end of the sheath 10 of the vessel wound closure device.

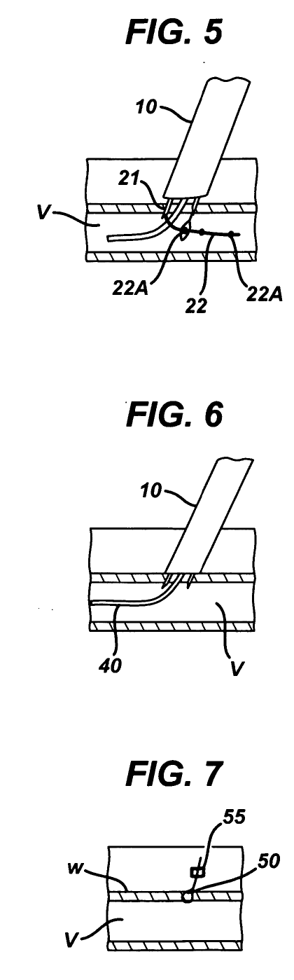

As shown in FIG. 5, the suture 22 is advanced beyond the needle 21 of the first vessel penetrating member 20 and into the vessel V. Ideally the naturally occurring blood flow in the direction of arrow a (FIG. 2) carries the suture 22 downstream and into the waiting snare 32. The suture 22 may includes knots 22A or other projections to help capture the suture 22 within the snare 32. Ideally, the operator can feel a resistance in at least one of the vessel penetrating members 20, 30 that indicates the suture 22 has been successfully captured within the snare 32. Thereafter, as shown in FIG. 6, the snare 32 and the suture 22 captured therein are withdrawn from the vessel V by retraction of the second vessel penetrating member 30.

The resistance identifying the capture of the suture 22 within the snare 32 is often best felt as the second penetrating member 30 is being retracted. When such retraction of the second vessel penetrating member 30 does not result in resistance to indicate that the suture 22 has been successfully captured within the snare 32, then the snare 32 is re-deployed, as before, within the vessel V until successful capture of the suture 22 in the snare 32 is achieved. After retraction of the second vessel penetrating member 30 with the captured suture 22 and snare 32 is achieved, the first vessel penetrating member 20 is also retracted, leaving only the loose ends of the suture 22 and snare 32 along the extravascular surface of the vessel wall w of the vessel V adjacent the puncture site. The guidewire 40 may then be withdrawn in conventional manner.

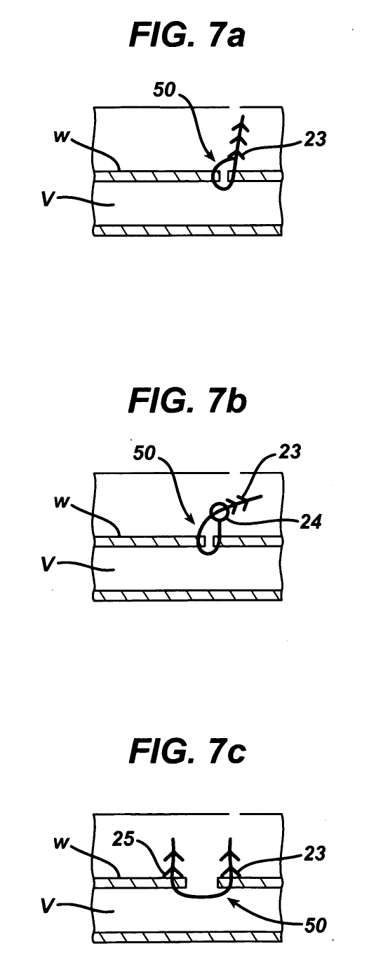

The suture 22 and snare 32 are then cut and tied, or otherwise secured, as shown, for example, in FIG. 7, in order to form a stitch 50 across the puncture wound of the vessel V. A one-way collar 55, knot, or other extravascular locking means, such as snap fittings, male-female ratcheting connections, and sutures and wires with a knot pushing device, or the like, could be slid down extravascularly over the suture 22 and snare 32 and tamped down in order minimize vessel puckering as the suture 22 and snare 32 are drawn to tighten and form the stitch 50 across the puncture site.

Alternatively, as shown in FIG. 7a, the suture 22 could include barbs 23, one of which that would embed within the outer surface of the vessel after the suture 22 has been withdrawn from the vessel by the snare 32 and then tightened to form the stitch 50 across the puncture site. The barbs 23 would permit one way transgression of the suture 22 through the vessel wall w in order to enter the vessel V, as described above, and would permit further one way transgression of the suture 22 through the vessel wall w in order to exit the vessel V when captured within the snare 32. Tightening of the suture 22 after sufficient withdrawal of the suture 22 from the vessel V by the snare 32, as before, would cause one barb 23 to expand extravascularly against the vessel wall w, whereafter the suture 22 is tied or otherwise secured to form the stitch 50 as before.

A still further alternative, as shown in FIG. 7b, provides a mesh or other fibrous structure 24 at the distal end of the suture 22, through which mesh or other fibrous structure 24 the suture 22 is passed and then tightened across the wound until one of the barbs 23 expands against the mesh or other fibrous structure 24. In this way, tying or other knotting of the suture to create the stitch 50 across the vessel is minimized, or ideally eliminated, while still providing an effective stitch across the vessel wound. In any case, once tightened and secured as desired across the vessel wound, the suture 22 is cut to leave the stitch 50 in place across the vessel wound. A cutting member, such as a guillotine-like cutting member 60 (FIG. 1) having a blade 61, a backing plate 62 and a drawstring 63 connected to one of the blade 61 or backing plate 62 for severing excess suture 22 after the stitch 50 is in place. Ideally, such cutting member is integrated with the vessel wound closure device 1, but may be separately deployed through the vessel wound closure device 1, in order to cut the excess suture.

A still further alternative, as shown in FIG. 7c, provides a suture 22 having one set of barbs 23 facing in one direction at a distal portion of the suture 22, and another set of barbs 25 facing in an opposite direction along a portion of the suture 22 spaced apart from the barbs 23. The spacing of the barbs 25 from the barbs 23 is preferably a sufficient distance such that the barbs 23 along the distal portion of the suture 22 penetrate through the vessel wall w to gain entry into the vessel and penetrate through the vessel wall w after capture by the snare 32 to exit the vessel, whereas the barbs 25 never pass through the vessel wall w or enter the vessel V. In this case, after the suture 22 is captured and withdrawn from the vessel V by the snare 32, the distal portion of the suture 22 is drawn until a barb 25 expands against the outer surface of the vessel wall w. The distal portion of the suture 22 is then further drawn to tighten the suture 22 across the wound sufficiently to form the stitch 50 thereacross as one of the barbs 23 also expands against the outer surface of the vessel wall w. Tying is likewise minimized, or ideally eliminated, using this technique as the expanded barbs 23, 25 anchor the stitch 50 in place across the vessel wound. Again, excess suture is cut as before.

Collagen or other thrombogenic material can be incorporated into the locking means to aid hemostasis at the puncture site where such a locking means is used. The collar 55, knot, or locking means is preferably bioabsorbable to accommodate resorption thereof in vivo. The collagen or other thrombogenic material may include therapeutic agents such as procoagulants, antimicrobial agents, anesthetics, or the like.

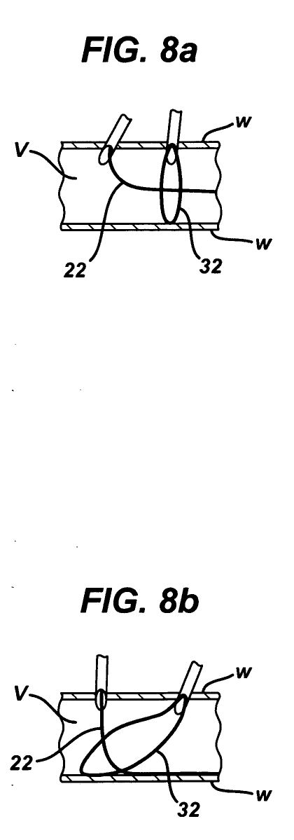

FIGS. 8a and 8b illustrate various options for positioning the snare 32 relative to the suture 22 and vessel V, wherein that option illustrated in FIG. 8b is preferred. In FIG. 8a, for example, the snare 32 is advanced from the second vessel penetrating member 30 so as to be generally perpendicularly situated between the vessel walls w of the vessel V. FIG. 8b, on the other hand, illustrates the snare 32 advanced from the second vessel penetrating member 30 so as to have a portion of the snare 32 lying generally beneath the upstream needle 21 of the first vessel penetrating member 20, from which the suture 22 is advanced.

Preferably the needles 21, 31 of the respective first and second vessel penetrating members 20, 30 are as small as possible in order to limit damage to the vessel wall. Such needles generally 18 gauge to 22 gauge needles, and preferably 21 gauge needles. The outer diameter of 21 gauge needles is approximately 0.032 inches. Of course, the artisan will readily appreciate that needles of other sizes are readily usable with the vessel wound closure device described herein. Similarly, the materials that comprise the sutures or snare are preferably biocompatible materials exhibiting high tensile and knot strength, although other materials may also be used with the vessel wound closure device described herein.

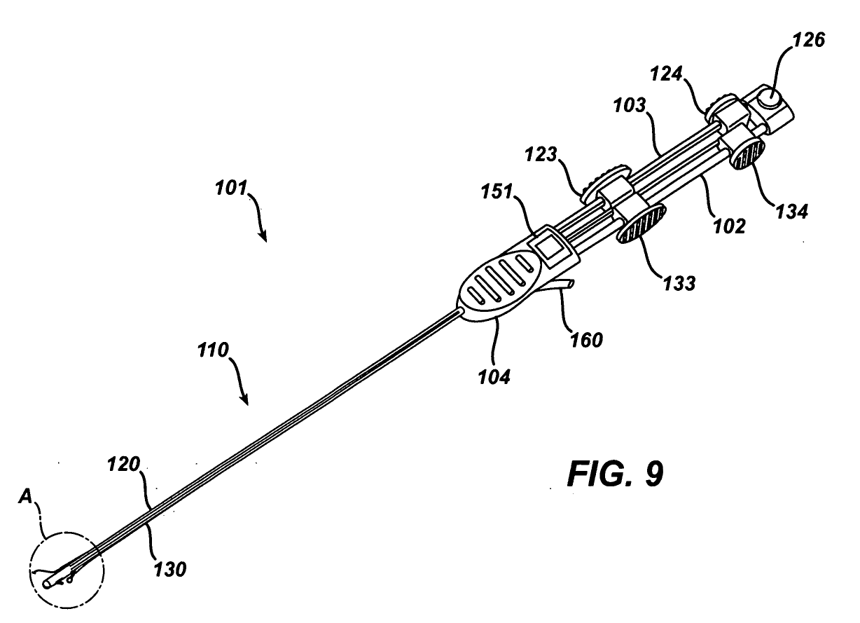

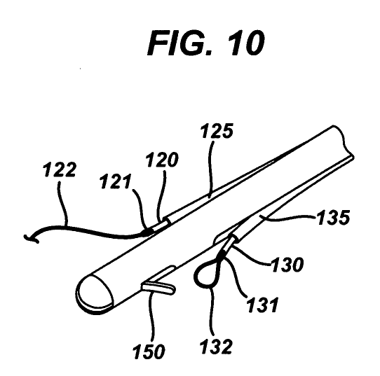

FIGS. 9-10 illustrate another embodiment of a vessel wound closure device 101 according to the invention. The vessel wound closure device 101 of FIGS. 9-10 may be inserted within an introducer already in place from a preceding procedure, or may be inserted over a guidewire after removal of an introducer from a preceding procedure. Emplacement and use of the vessel wound closure device 101 is generally the same unless otherwise specified herein, regardless of whether the device 101 is inserted within a preexisting introducer or over a guidewire wherein the preexisting introducer has been removed.

FIG. 9 illustrates the vessel wound closure device 101 comprising a sheath 110 having a distal end and a proximal end, wherein the distal end is understood as furthest from the operator and the proximal end is understood as closest to the operator. The distal end of the vessel wound closure device 101, encircled in inset A of FIG. 9, is shown in greater detail in FIG. 10.

The portion of the wound closure device 101 proximal of the sheath 110 comprises a pair of generally parallel rails 102, 103 upon which ride slides 123, 124, 133 and 134 that regulate movement of various components discussed in further detail below. A grip 104, having an exterior surface and an interior channel, provides a rest for an operator's hand or digits along the exterior surface thereof, and provides a transition piece in which the various components passing therethrough the interior of said grip 104 are appropriately connected to extend proximal to distal and are regulated by the slides 123, 124, 133 and 134

Referring to FIGS. 9 and 10, the sheath 110 further comprises a first vessel penetrating member 120 having a needle 121 and a suture 122 extending therethrough, similar to the earlier described embodiments herein. The sheath 110 further comprises a second vessel penetrating member 130 having a needle 131 and a snare 132 extending therethrough, also similar to the earlier described embodiments herein. The distal end of the vessel wound closure device 101 further comprises a stabilizer foot 150 that is flush with the exterior surface of the sheath 110 when retracted, but that projects laterally out from the exterior surface of the 110 when deployed. Although shown as a generally solid component in FIGS. 9 and 10, the stabilizer foot 150 may also be configured as a non-solid component, such as a spiral or other web, mesh or wire configuration, or combinations thereof, as the artisan should readily appreciate, provided such stabilizer foot 150 is retractable and deployable as described herein and comprised of biocompatible materials known or later developed in the art that are suitable for use within the vasculature of a patient. A foot actuating lever 151 provided in the grip 104 regulates movement of the stabilizer foot 150 in conventional manner via a cable or other linkage system.

A first vessel penetrating member slide 123 is connected to a proximal portion of the first vessel penetrating member 120 and regulates movement thereof. A suture slide 124 is connected to a proximal end of the suture 122 and regulates movement thereof. A distal end of the introducer 110 also includes a first exit port 125, through which the first vessel penetrating member 120 and suture 122 are manipulated. A suture storage unit 126 may be provided at a proximal end of the vessel wound closure device 101, if desired, in order to spool suture material therefrom as needed in practice.

A second vessel penetrating member slide 133 is connected to a proximal portion of the second vessel penetrating member 130 and regulates movement thereof. A snare slide 134 is connected to a proximal end of the snare 132 and regulates movement thereof. A distal end of the sheath 110 also includes a second exit port 135, through which the second vessel penetrating member 130 and snare 132 are manipulated.

For those instances when the vessel wound closure device 101 is inserted over a guidewire, and not within a preexisting introducer from a preceding procedure, then positioning of the device 101 appropriately within the vessel may be achieved by the identification of blood flashback. Accordingly, a flashback port 160 is provided on the grip 104, for example. Of course, the flashback port 160 could instead be located elsewhere along the introducer in the artisan's discretion.

In practice, the vessel wound closure device 101 is inserted into a vessel through a preexisting introducer from a preceding procedure, or over a guidewire extending into a vessel after removal of the preexisting introducer. In the former case, positioning of the vessel wound closure device 101 within the vessel is determined in conventional manner relying on the dimensions, position or markings of the preexisting introducer, whereas in the latter case, flashback of blood from the vessel through the flashback port 160 determines when the vessel wound closure device 101 is appropriately positioned within the vessel.

Once the distal end of the sheath 110 of the vessel wound closure device 101 is appropriately positioned within the vessel, then remaining procedures are generally as follows regardless of whether the device 101 is inserted through a preexisting introducer or over a guidewire, unless otherwise indicated herein. First, the stabilizer foot activation lever 160 is activated to deploy the stabilizer foot 150 within the vessel. Then, the entire vessel wound closure device 101 is retracted until the stabilizer foot 150 abuts the interior wall of the vessel.

The second vessel penetrating member slide 133 is then pushed distally to deploy the second vessel penetrating member 130. The snare slide 134 moves in unison with the second vessel penetrating member slide 133 at this point. Once the second vessel penetrating member 130 is deployed, the second vessel penetrating member slide 133 is locked and the snare slide 134 is slid independently further distally until the snare 132 is deployed beyond the needle 131 and within the vessel.

Next, the first vessel penetrating member slide 123 is pushed distally to deploy the first vessel penetrating member 120. The suture slide 124 moves in unison with the first vessel penetrating member slide 123 at this point. Once the first vessel penetrating member 120 is deployed within the vessel, then the first vessel penetrating member 123 is locked and the suture slide 124 is slid independently further distally until the suture 122 is deployed beyond the needle 121 and within the vessel.

As in earlier embodiments, the snare 132 is deployed downstream of the suture 122 to enhance the capture of the suture by the snare. The suture 122 may further comprise knots or other projections, as in earlier described embodiments, also to enhance capture thereof by the snare. Capture of the suture 122 by the snare 132 is identified by resistance felt by an operator as the snare slide 134 is slid proximally to retract the snare 132, ideally with the suture 122 captured thereby. Should resistance not be experienced by the operator, then the snare slide 133 is moved distally again to redeploy the snare 132 as before. This process repeats itself until the suture 122 is successfully captured by the snare 132.

Once the suture 122 is captured by the snare 132, then the snare 132 is retracted by proximally sliding of the snare slide 134, and the second vessel penetrating member 130 is retracted by proximally sliding of the slide 133. The first vessel penetrating member 120 is also retracted by proximally sliding of the slide 123. The stabilizer foot 150 is de-activated and returned to its non-deployed state flush with the exterior surface of the sheath 110 by the stabilizer foot lever 151. The entire vessel wound closure device 101 is then removed from the vessel through the incision tract, and loose ends of the of suture are knotted as in earlier described embodiments to form a stitch across the puncture site.

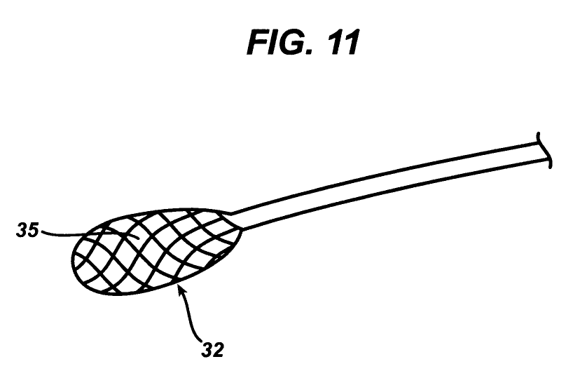

FIG. 11 illustrates the looped end of a snare 32 or 132. The looped end of the snare 32 or 132 is shown in FIG. 11 as further comprising a mesh basket 35. Where used, the mesh basket 35 enables the capture of more suture 22 or 122 therein, whereafter the captured suture is withdrawn to create the stitch across the puncture wound as described in the various embodiments herein.

Of course, although the various embodiments described herein are directed to creating a single stitch across a vessel wound by deployment of a suture and snare from a first vessel penetrating and a second vessel penetrating member, various other embodiments could comprise creating more than a single stitch across the vessel wound. Such embodiments may comprise additional vessel penetrating members, sutures and snares, for example, that are otherwise deployable as described herein.

The various exemplary embodiments of the invention as described hereinabove do not limit different embodiments of the systems and methods of the invention. The material described herein is not limited to the materials, designs or shapes referenced herein for illustrative purposes only, and may comprise various other materials, designs or shapes suitable for the systems and methods described herein, as should be appreciated by the artisan.

While there has been shown and described what is considered to be preferred embodiments of the invention, it will, of course, be understood that various modifications and changes in form or detail could readily be made without departing from the spirit or scope of the invention. It is therefore intended that the invention be not limited to the exact forms described and illustrated herein, but should be construed to cover all modifications that may fall within the scope of the appended claims.

What is claimed is:

1. A vessel wound closure device for use with a vessel wound comprising:
a sheath having a proximal end and a distal end;
a first hollow vessel penetrating member associated with the sheath and a second hollow vessel penetrating member associated with the sheath, wherein the first and second vessel penetrating members are movable from a first position to a second position beyond the distal end of the sheath to penetrate through a vessel wall adjacent the vessel wound;
a suture slidably housed within the first hollow vessel penetrating member; and
a single filament snare loop slidably housed within the second hollow vessel penetrating member, wherein the snare loop is positionable to capture the suture when the first and second vessel penetrating members are in the second position.

2. The vessel wound closure device of claim 1, further comprising a needle at a distal end of the first hollow vessel penetrating member, and a needle at a distal end of the second hollow vessel penetrating member.

3. The vessel wound closure device of claim 1, further comprising a bioabsorbable locking means applied to the suture and snare loop.

4. The vessel wound closure device of claim 3, wherein the locking means is one of a collar, a knot, a snap-fitting, a ratcheting connection, or sutures and wires.

5. The vessel wound closure device of claim 3, further comprising a thrombogenic material incorporated into the locking means.

6. The vessel wound closure device of claim 1, further comprising a guide associated with the sheath for each respective first and second vessel penetrating member.

7. The vessel wound closure device of claim 1, wherein the suture further comprises knots or other projections.

8. The vessel wound closure device of claim 11, wherein at least one of the first and second vessel penetrating members comprises a blood flashback conduit configured to identify when the vessel has been penetrated by the respective at least one of the first and second vessel penetrating members.

9. The vessel wound closure device of claim 1, wherein the second hollow vessel penetrating member is located downstream of the first hollow vessel penetrating member.

10. The vessel wound closure device of claim 1, wherein the suture further comprises at least one barb at a distal end thereof.

11. The vessel wound closure device of claim 1, wherein the suture further comprises a fibrous structure at a distal end of the suture and at least one barb along the suture.

12. A method for closing a vessel wound in a vessel of a patient, the method comprising:
deploying a first hollow vessel penetrating member and a second hollow vessel penetrating member through a vessel wall and into the vessel adjacent the vessel wound;
deploying a suture from the first hollow vessel penetrating member;
deploying a single filament snare loop from the second hollow vessel penetrating member; and
capturing the suture within the single filament snare loop within the vessel.

13. The method of claim 12, wherein the first and second vessel penetrating members are associated with a sheath and deployed through an introducer.

14. The method of claim 12, wherein the first and second vessel penetrating members are associated with a sheath and deployed over a guidewire.

15. The method of claim 14, further comprising determining the location of the first and second vessel penetrating members within the vessel by providing flashback of blood from the vessel through at least one of the first and second hollow vessel penetrating members.

16. The method of claim 12, further comprising orienting the second hollow vessel penetrating member downstream of the first hollow vessel penetrating member within the vessel.

17. The method of claim 16, wherein capture of the suture within the snare loop is determined by sensing resistance in at least one of the first and second vessel penetrating members.

18. The method of claim 16, wherein the snare loop is deployed to be approximately perpendicularly situated between vessel walls of the vessel.

19. The method of claim 16, wherein the snare loop is deployed to have a portion thereof approximately beneath the first hollow vessel penetrating member.

20. The method of claim 16, wherein the suture and snare loop are withdrawn from the vessel through one of the first and second hollow vessel penetrating members and extravascularly secured.

21. The method of claim 20, wherein extravascularly securing the suture and snare loop comprises sliding a bioabsorbable locking means over the suture and snare loop.

22. The method of claim 20, wherein extravascularly securing the suture and snare loop further comprises providing at least one barb on a distal portion of the suture that deflects against an outer surface of the vessel wall when the suture is tightened.

23. The method of claim 20, wherein extravascularly securing the suture and snare loop further comprises providing a fibrous structure at distal end of the suture and at least one barb that penetrates through and deflects so that it is positioned against the fibrous structure after capture and withdrawal of the suture from the vessel by the snare loop.

24. A vessel wound closure device comprising:
a sheath having a proximal end and a distal end;
a thumb grip between the proximal end and the distal end of the sheath;
the sheath extending distally from the thumb grip and having a first exit port, a second exit port, and a movable stabilizer foot at a distal end thereof;
a first hollow vessel penetrating member extending through the sheath;
a second hollow vessel penetrating member extending through the sheath;
a slidable suture extending through the first hollow vessel penetrating member;
a slidable snare loop extending through the second hollow vessel penetrating member; and a first rail extending proximally from the thumb grip, the first rail having a first slide mounted thereon for regulating movement of the first hollow vessel penetrating member and a second slide mounted thereon for regulating movement of the suture.

25. The vessel wound closure device of claim 24, further comprising a needle at a distal end of each of the first and the second hollow vessel penetrating members.

26. The vessel wound closure device of claim 24, wherein the thumb grip further comprises a stabilizer foot activation lever for deploying the stabilizer foot from a retracted position to a deployed position.

27. The vessel wound closure device of claim 26, wherein the thumb grip further comprises a flashback port through which flashback blood from the vessel and through at least one of the respective first and second hollow vessel penetrating members flows to identify the location of the vessel penetrating members in the vessel.

28. The vessel wound closure device of claim 24, further comprising a suture storage unit.

29. The vessel wound closure device of claim 24, further comprising a locking means for securing the suture and snare loop after capture and withdrawal thereof from the vessel to form a stitch across the vessel wound.

30. The vessel wound closure device of claim 24, further comprising:
a second rail, generally parallel to the first rail, extending proximally from the thumb grip, the second rail having a third slide mounted thereon for regulating movement of the second hollow vessel penetrating member and a fourth slide mounted thereon for regulating movement of the snare loop, wherein the suture deployed from the first exit port is captured within the snare loop deployed from the second exit port and withdrawn and secured extravascularly to form a stitch across the vessel wound.

31. A method of closing a vessel wound in a vessel, the method comprising:
deploying a first hollow vessel penetrating member and a second hollow vessel penetrating member through a vessel wall and into the vessel adjacent the vessel wound;
deploying a suture into the vessel from the first hollow vessel penetrating member;
deploying a snare loop into the vessel from the second hollow vessel penetrating member; and capturing the suture within the snare loop.

32. The method of claim 31, further comprising regulating deployment of the first hollow vessel penetrating member by a first slide and regulating deployment of the suture by a second slide, wherein the first slide and the second slide are on a common rail.

33. The method of claim 32, further comprising stopping movement of the first slide after the first hollow vessel penetrating member is deployed within the vessel and then deploying the suture by further sliding of the second slide.

34. The method of claim 33, wherein the first hollow vessel penetrating member proceeds through a first exit port and includes a needle to penetrate a vessel wall adjacent the vessel wound.

35. The method of claim 34, further comprising regulating deployment of the second hollow vessel penetrating member by a third slide and regulating deployment of the snare loop by a fourth slide, wherein the third slide and fourth slide are on a common rail.

36. The method of claim 35, further comprising stopping movement of the third slide after the second hollow vessel penetrating member is deployed within the vessel and deploying the snare loop within the vessel by further sliding of the fourth slide.

37. The method of claim 36, wherein the second hollow vessel penetrating member proceeds through a second exit port and includes a needle to penetrate the vessel wall adjacent the vessel wound.

38. The method of claim 36, further comprising orienting the second hollow vessel penetrating member to be deployed downstream of the first hollow vessel penetrating member.

39. The method of claim 38, further comprising withdrawing the captured suture and snare loop from the vessel by proximal sliding of the fourth slide.

40. The method of claim 39, further comprising withdrawing the second hollow vessel penetrating member from the vessel by proximal sliding of the third slide.

41. The method of claim 40, further comprising extravascularly securing the captured suture and snare loop with a bioabsorbable locking means.

42. The method of claim 41, further comprising deploying a stabilizer foot prior to identifying the location of the first and second exit ports within the vessel prior to deployment of the first and second hollow vessel penetrating members.

43. The method of claim 41, further comprising identifying the location of the first and second exit ports within the vessel prior to deployment of the first and second hollow vessel penetrating members by flashback of blood from the vessel.
